(19) United States
(12) Patent Application Publication
(10) Pub. No.: US 2026/0021032 A1
(43) Pub. Date: Jan. 22, 2026

(54) Therapeutic Scalp and Skin Care Composition

(71) Applicant: Christopher Davis, Cincinnati, OH (US)

(72) Inventor: Christopher Davis, Cincinnati, OH (US)

(21) Appl. No.: 19/266,734

(22) Filed: Jul. 11, 2025

Related U.S. Application Data
(60) Provisional application No. 63/672,801, filed on Jul. 18, 2024.

(51) Int. Cl. A61K 8/92; A61K 8/9794; A61Q 19/00

(52) U.S. Cl. CPC A61K 8/922; A61K 8/9794; A61Q 19/00; A61Q 19/007

(57) ABSTRACT

The present invention relates to a scalp and skin irritation prevention composition in the form of a lotion, spray, or ointment for relieving scalp and skin irritation, and dryness and promoting hair growth. The composition is novel, easy to use, and made from natural and organic ingredients. The composition includes aloe vera, peppermint oil, lavender oil, rosemary oil, Virginia olive oil, organic avocado oil, grapeseed oil, sunflower oil, safflower oil, geranium oil, marjoram oil, lemongrass oil, tea tree oil, cedarwood oil, and argan oil. The composition can be applied to the scalp or other areas of the body for moisturizing, soothing inflammation, and repairing damaged hair and skin. The composition is suitable for all genders and age groups, offering a natural solution to common scalp issues such as dandruff, irritation, and hair thinning.

### CROSS-REFERENCE TO RELATED APPLICATION

The present application claims priority to, and the benefit of, U.S. Provisional Application No. 63/672,801, which was filed on Jul. 18, 2024, and is incorporated herein by reference in its entirety.

### FIELD OF THE INVENTION

The present invention generally relates to the field of hair and skin care products. More specifically, the present invention relates to a novel scalp and skin irritation prevention composition designed to address issues such as scalp and skin dryness, inflammation, dandruff, and hair thinning. The composition combines natural oils and essential oils with a soothing aloe vera base to provide deep moisturization and scalp treatment. The composition can be formulated as a lotion, spray, or ointment, and can be easily applied to the scalp or other areas of the body. Accordingly, this disclosure makes specific reference thereto the present invention. Nonetheless, it is to be appreciated that aspects of the present invention are also equally applicable to other like applications, devices and methods of manufacture.

### BACKGROUND

By way of background, many people suffer from dry skin and scalp inflammation. Dry skin on the scalp often leads to dandruff, which, when left untreated, can build up and cause further irritation. Bacteria and other microbes can thrive on the dry skin exacerbating the scalp's irritation and inflammation. As a result, individuals may experience itching, redness, and flaking, which can worsen over time if not properly addressed.

Chronic scalp dryness and dandruff can compromise the integrity of the skin on the scalp, potentially leading to broken skin, which is not only uncomfortable but also more prone to infections. Furthermore, the lack of moisture in both the scalp and hair can lead to dry, brittle hair that is prone to breakage. Over time, this can contribute to early hair thinning and balding.

Many individuals try good-quality creams or gels to create a lubricated surface to reduce itching, redness, and flaking. Also, people use cold press and different lotions to moisturize their skin and scalp. However, even after using such methods, people feel uncomfortable and disappointed with dry, rough skin and hair. People desire a more organic and effective topical composition that can moisturize, soothe inflamed skin, and promote hair growth while providing relief from scalp irritation.

Therefore, there exists a long-felt need in the art for a composition that effectively addresses dry skin, inflammation, and irritation on the scalp. There is also a long-felt need for a composition that provides relief from dandruff buildup and soothes the discomfort caused by dry, itchy scalp and skin conditions. Additionally, there is a long-felt need for a scalp treatment that prevents bacterial and fungal infections, which commonly arise due to dandruff buildup. Moreover, there is a long-felt need for a composition that promotes hair growth by creating a healthy scalp environment. Further, there is a long-felt need for a composition that moisturizes skin and scalp deeply without leaving a greasy residue. Furthermore, there is a long-felt need in the art for a natural, organic scalp treatment solution that is safe for all skin types, including sensitive skin. Finally, there is a long-felt need in the art for a convenient scalp treatment that can be applied topically in various forms such as lotions, sprays, or ointments, making it easy to use in daily hair care routines.

The subject matter disclosed and claimed herein, in one embodiment thereof, comprises a scalp irritation prevention composition designed to relieve dry scalp, inflammation, and promote hair growth. The composition includes aloe vera, peppermint oil, lavender oil, rosemary oil, Virginia olive oil, organic avocado oil, grapeseed oil, sunflower oil, safflower oil, geranium oil, marjoram oil, lemongrass oil, tea tree oil, cedarwood oil, and argan oil. The composition can be formulated as a lotion, spray, or ointment for easy topical application. When applied to the scalp or skin, the composition moisturizes deeply, reduces dandruff and irritation, and enhances scalp health to support hair growth. The combination of essential oils and natural moisturizers provides protection from scalp infections and helps repair damaged skin and hair, making the composition highly effective for people suffering from dry, itchy scalp or thinning hair.

In this manner, the novel scalp irritation prevention composition of the present invention accomplishes all of the foregoing objectives and provides users with a safe, natural, and effective solution for maintaining a healthy scalp, hair growth and moisturized skin. The composition soothes scalp irritation and dryness and also prevents hair thinning and breakage by deeply moisturizing the hair and scalp. The composition can be conveniently applied as part of a daily routine in the form of a lotion, spray, or ointment, offering users flexibility and ease of use.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a scalp and skin irritation prevention composition for preventing scalp irritation, improving hair growth, and moisturizing skin and scalp. The composition includes aloe vera, peppermint oil, lavender oil, rosemary oil, Virginia olive oil, organic avocado oil, grapeseed oil, sunflower oil, safflower oil, geranium oil, marjoram oil, lemongrass oil, tea tree oil, cedarwood oil, and argan oil, wherein the composition can be in the form of a lotion, spray, or ointment. The composition can be used by people of all ages and genders and is applied topically to the scalp or other areas of the body for therapeutic purposes.

In a further embodiment of the present invention, a method for preventing scalp and skin irritation and promoting hair growth comprises the steps of applying the composition to an individual's scalp or skin, the composition comprising: 25-30% aloe vera, 1-2% peppermint oil, 5-7% lavender oil, 1-3% rosemary oil, 12-18% Virginia olive oil, 15-20% organic avocado oil, 1-3% grapeseed oil, 10-15% sunflower oil, 5-8% safflower oil, 2-3% geranium oil, 1-2% marjoram oil, 2-3% lemongrass oil, 1-2% tea tree oil, 1-2% cedarwood oil, and 1-2% argan oil.

In a further embodiment of the present invention, a composition for use as a scalp irritation prevention lotion comprises, by weight of the composition: 20-25% aloe vera, 0.5-1.5% peppermint oil, 3-5% lavender oil, 1-2% rosemary oil, 15-20% Virginia olive oil, 16-18% organic avocado oil, 1-2% grapeseed oil, 12-15% sunflower oil, 2-6% safflower oil, 2-5% geranium oil, 1-2% marjoram oil, 2-3% lemongrass oil, 1-2% tea tree oil, 1-2% cedarwood oil, and 1-2% argan oil.

The composition of the present invention is made from organic, natural ingredients, and does not include harmful chemicals, making it suitable for sensitive skin and for individuals with scalp conditions such as dandruff, seborrheic dermatitis, and psoriasis. The composition is preferably transparent or translucent and can be topically applied to the skin or scalp as a lotion, spray, or ointment.

The advantage of the scalp and skin irritation prevention composition of the present invention is that it provides users with an improved product designed to protect the scalp from irritation, promote hair growth, and provide long-lasting hydration to the skin and scalp. The ingredients work synergistically to achieve moisturizing, soothing, and healing properties. The composition can be applied at room temperature and provides relief from dry or irritated scalp while supporting overall scalp health.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a diagrammatic representation of the components of the scalp and skin irritation prevention composition of the present invention;

FIG. 2 illustrates one potential embodiment of the system used for manufacturing the composition of the present invention in accordance with the disclosed structure;

FIG. 3 illustrates an embodiment of the container in which the scalp and skin irritation prevention composition 100 can be available for consumers; and

FIG. 4 illustrates a flow chart showing steps used in manufacturing the therapeutic composition of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a composition that effectively addresses dry skin, inflammation, and irritation on the scalp. There is also a long-felt need for a composition that provides relief from dandruff buildup and soothes the discomfort caused by dry, itchy scalp and skin conditions. Additionally, there is a long-felt need for a scalp treatment that prevents bacterial and fungal infections, which commonly arise due to dandruff buildup. Moreover, there is a long-felt need for a composition that promotes hair growth by creating a healthy scalp environment. Further, there is a long-felt need for a composition that moisturizes skin and scalp deeply without leaving a greasy residue. Furthermore, there is a long-felt need in the art for a natural, organic scalp treatment solution that is safe for all skin types, including sensitive skin. Finally, there is a long-felt need in the art for a convenient scalp treatment that can be applied topically in various forms such as lotions, sprays, or ointments, making it easy to use in daily hair care routines.

The present invention, in one exemplary embodiment, is a scalp and skin irritation prevention composition for preventing scalp irritation, improving hair growth, and moisturizing skin and scalp. The composition includes aloe vera, peppermint oil, lavender oil, rosemary oil, Virginia olive oil, organic avocado oil, grapeseed oil, sunflower oil, safflower oil, geranium oil, marjoram oil, lemongrass oil, tea tree oil, cedarwood oil, and argan oil, wherein the composition can be in the form of a lotion, spray, or ointment. The composition can be used by people of all ages and genders and is applied topically to the scalp or other areas of the body for therapeutic purposes.

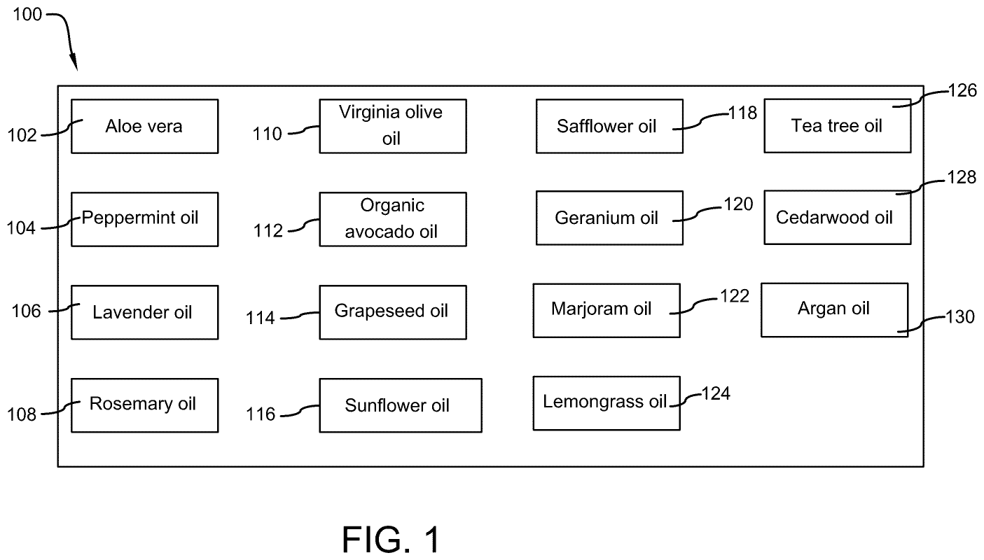

Referring initially to the drawings, FIG. 1 illustrates a diagrammatic representation of the components of the scalp and skin irritation prevention composition 100 of the present invention. The scalp irritation prevention composition 100 of the present invention is a topical lotion designed for use on hair and skin. The composition 100 is made primarily from fresh aloe vera combined with a blend of various organic oils and essential oils. The composition 100 can be applied to the scalp or other areas of the body for therapeutic treatment. All the ingredients provide a unique quality and increase the effectiveness of the scalp irritation prevention composition 100 in moisturizing, stimulating hair growth, soothing, and healing.

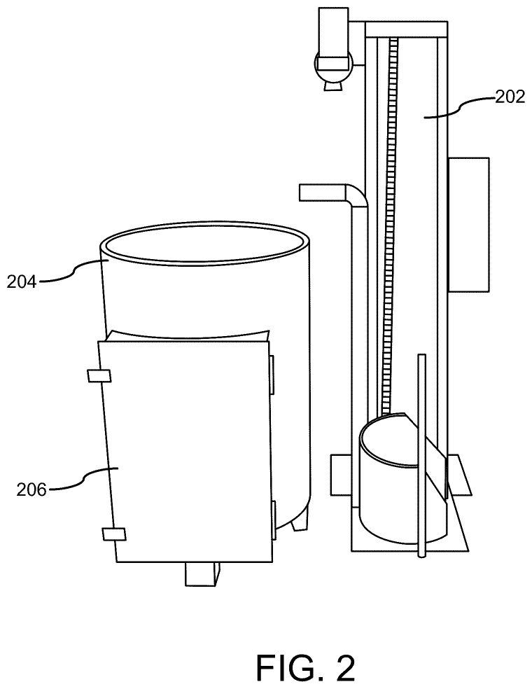

Aloe vera 102 forms the base of the composition 100, and is preferably blended and filtered to remove any solid particles therein to create a smooth liquid. Aloe vera 102 has hydrating properties and helps lock in moisture, providing long-lasting hydration without leaving a greasy residue. Aloe vera 102 also contains aloins and polysaccharides that have soothing, anti-inflammatory effects and is used for treating irritated skin, soothing sunburn, and reducing scalp irritation or itching. Aloe vera 102 contains proteolytic enzymes that help repair dead skin cells on the scalp, allowing for improved hair follicle health. In the preferred embodiment, fresh aloe vera 102 is blended in an industrial blender to break down the natural gel-like structure thereof into a smooth, liquid form as illustrated in FIG. 2. Aloe vera 102 also acts as a carrier for the essential oils and organic oils added to the formulation. Aloe vera 102 also helps to evenly distribute the oils across the skin and hair. Aloe vera 102 can be extracted directly from the Aloe vera plant or any commercially available aloe vera extracts.

The composition 100 also includes a plurality of organic essential oils for providing relief from dry scalp and skin. The composition 100 may be comprised of a peppermint oil 104 containing menthol that stimulates blood circulation in the scalp and soothes irritated skin. The cooling sensation provided by menthol has a soothing effect on irritated skin and scalp. Peppermint oil 104 also has antibacterial, antifungal, and antiviral properties and is useful for treating scalp conditions like dandruff, seborrheic dermatitis, and other skin infections. Peppermint oil 104 further helps to decongest clogged pores and reduce the appearance of acne, blackheads, and other impurities. In the preferred embodiment, Peppermint oil 104 is extracted through steam distillation of the leaves of the peppermint plant (Mentha piperita).

Lavender oil 106 may also be included in the composition 100 and provides calming properties and promotes hair growth. Specifically, Lavender oil 106 has natural antiseptic and antifungal qualities and helps keep the scalp and skin clean and free from bacteria or fungal infections. Lavender oil 106 also provides hydration to both skin and hair to lock in moisture and keep the skin soft and the hair conditioned, which reduces frizz and split ends. Preferably, Lavender oil 106 is extracted through steam distillation of the lavender plant's flowers.

Rosemary oil 108 may also be included to stimulate hair growth, improve scalp circulation, and treat dandruff. Rosemary oil 108 is preferably extracted via steam distillation from rosemary leaves. Rosemary oil 108 contains natural antioxidants that protect hair and skin from free radical damage, which can lead to premature aging of the scalp and weaken hair structure over time. Rosemary oil 108 used in the composition 100 includes potent active compounds like camphor, cincole, and alpha-pinene, which are responsible for stimulating hair growth and providing antimicrobial properties.

Virginia olive oil 110 may also be included and is rich in vitamins A and E, it hydrates deeply, repairs split ends and protects against environmental damage. Also, Virginia olive oil 110 has a high content of monounsaturated fatty acids (mainly oleic acid) and functions as a natural emollient. Virginia olive oil is extracted through cold pressing from olives and retains all its natural vitamins, antioxidants, and fatty acids.

Organic avocado oil 112 may be included and includes fatty acids and vitamins (especially vitamin E). Organic avocado oil 112 nourishes, protects, and strengthens both hair and skin. Avocado oil helps repair damaged hair, especially hair that has been weakened by heat styling, chemical treatments, or environmental exposure. Avocado oil also stimulates collagen production which can help strengthen hair and improve skin elasticity. Avocado oil has a high concentration of oleic acid which makes it an excellent emollient that helps retain moisture in both hair and skin.

Grapeseed oil 114 may be included in one embodiment and is rich in antioxidants and linoleic acid and conditions without clogging pores, thereby is useful for oily or acne-prone skin. The Grapeseed oil 114 is extracted from grape seeds via cold-pressing and functions as a carrier for other essential oils. The Grapeseed oil 114 used in the composition 100 contains approximately 70% linoleic acid to soothe and heal the skin. Linoleic acid helps strengthen the skin barrier, reducing moisture loss and promoting a more even skin texture.

In one embodiment, sunflower oil 116 is included and is rich in vitamin E and offers antioxidant protection and locks in moisture without making the skin or hair greasy. Sunflower oil 116 moisturizes hair and skin, reduces frizz, and promotes a healthy scalp. Safflower oil 118 is high in oleic acid and reduces scalp dryness and improves skin smoothness. Safflower oil 118 also hydrates skin and hair, reduces inflammation, and improves skin texture. Geranium oil 120 balances oil production in both hair and skin, reduces inflammation, and enhances circulation. Geranium oil 120 also helps regulate sebum levels and is ideal for oily or acne-prone skin and promotes balanced scalp health.

Marjoram oil 122 may be included for its powerful anti-inflammatory and antioxidant properties for soothing irritated or inflamed skin and scalp. Marjoram oil 122 helps alleviate conditions like eczema, psoriasis, and dermatitis by reducing redness and discomfort. Marjoram oil 122 also has mild antimicrobial and antiseptic properties which help keep the scalp clean and free from bacteria or fungal infections.

Lemongrass oil 124 may be included as a natural antiseptic for the scalp and skin, which helps clean and detoxify the scalp, while reducing excess oil and dandruff. Lemongrass oil 124 is extracted through steam distillation of lemongrass and offers a fresh scent and antimicrobial properties. Tea tree oil 126 has antifungal, and antibacterial properties and treats scalp conditions like dandruff and infections while also soothing inflamed skin. Tea tree oil 126 is steam distilled from the leaves of the tea tree.

Cedarwood oil 128 is included in one embodiment. Cedarwood oil 128 stimulates hair growth, treats scalp conditions, and balances oil production. Cedarwood oil 128 is extracted via steam distillation from cedarwood bark. Cedarwood oil has natural antifungal and antibacterial properties that help combat infections of the scalp, such as fungal dandruff or minor bacterial infections. Cedarwood oil helps regulate the production of sebum (natural scalp oils) making it beneficial for both oily and dry scalps. Argan oil 130 is rich in fatty acids and vitamin E and adds shine, reduces frizz, and provides deep conditioning for both skin and hair. Argan oil 130 is cold-pressed from the kernels of the argan tree.

The scalp and skin irritation prevention composition of the present invention is safe for humans and has been demonstrated to not irritate the skin even when used multiple times in a day and for a long period of time. It should be noted that all the ingredients work synergistically to enhance the overall efficacy of the composition 100. The composition of the present invention is useful as a pharmaceutical or an ayurvedic product and functions for all genders.

Referring to Table 1, there is disclosed a scalp irritation prevention composition 100 with a number of different components in varying quantities in order to achieve a desired result. The scalp and skin irritation prevention composition 100, in one potential embodiment, is provided as a transparent or translucent lotion. One such exemplary embodiment of the scalp irritation prevention composition 100 is provided as shown in Table 1.

TABLE 1

| | Composition in percentage by weight |
|---|---|
| Component | (% w/w) |
| Peppermint oil | 1-2 |
| Lavender oil | 5-7 |
| Rosemary oil | 1-3 |
| Virginia olive oil | 12-18 |
| Organic avocado oil | 15-20 |
| Grapeseed oil | 1-3 |
| Sunflower oil | 10-15 |
| Safflower oil | 5-8 |
| Geranium oil | 2-3 |
| Marjoram oil | 1-2 |
| Lemon grass oil | 2-3 |
| Tea tree oil | 1-2 |
| Cedarwood oil | 1-2 |
| Argon oil | 1-2 |
| Aloe vera | 25-30 |

Table 2, there is disclosed alternative scalp and skin irritation prevention composition 100 with a number of different components in varying quantities in order to achieve a desired result.

TABLE 2

| | Composition in percentage by weight |
|---|---|
| Component | (% w/w) |
| Peppermint oil | 0.5-1.5 |
| Lavender oil | 3-5 |
| Rosemary oil | 1-2 |
| Virginia olive oil | 15-20 |
| Organic avocado oil | 16-18 |
| Grapeseed oil | 1-2 |
| Sunflower oil | 12-15 |
| Safflower oil | 2-6 |
| Geranium oil | 2-5 |
| Marjoram oil | 1-2 |
| Lemon grass oil | 2-3 |
| Tea tree oil | 1-2 |
| Cedarwood oil | 1-2 |
| Argon oil | 1-2 |
| Aloe vera | 20-25 |

FIG. 2 illustrates one potential embodiment of the system used for manufacturing the composition of the present invention in accordance with the disclosed structure. As illustrated, the system 200 includes a blender 202 to break down the natural aloe vera gel to make a smooth liquid that retains the moisture and healing properties of the aloe vera extract. The blender 202 can be of any size and shape depending on the volume of the aloe vera gel to be processed. A fine mesh strainer 204 is used for removing any residual solid particles from the blended aloe vera extract. A mixer 206 is used for mixing different organic oils in the aloe vera juice and each oil is added based on their specific benefits for hair and skin care, thereby creating a comprehensive and synergistic composition. The composition 100 is made from organic, natural ingredients and is free from harsh chemicals, making it suitable for sensitive skin and scalps.

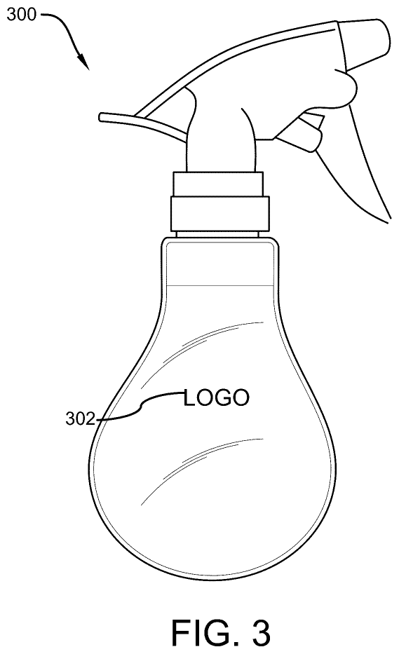

FIG. 3 illustrates an embodiment of the container in which the scalp and skin irritation prevention composition 100 can be available for consumers. In the present embodiment, the scalp and skin irritation prevention composition 100 is stored in a 300 spray unit and can be topically applied to any affected area by a user. In some embodiments, the composition 100 can also be made in the form of an ointment. A logo or indicia 302 can be disposed of on the unit 300 for marketing and branding purposes.

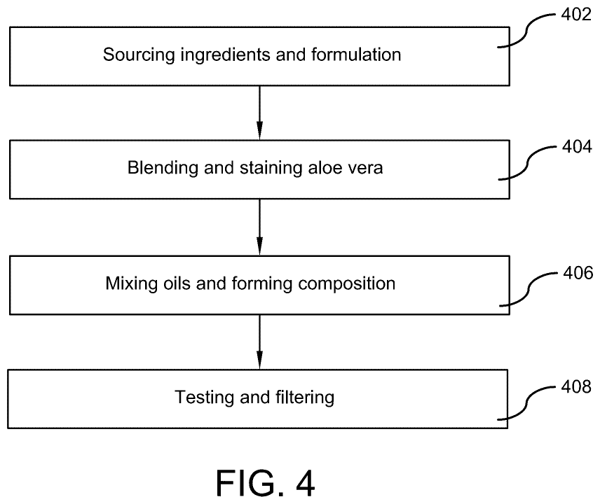

FIG. 4 illustrates a flow chart showing steps used in manufacturing the therapeutic composition of the present invention in accordance with the disclosed architecture. Initially, ingredients described in Tables 1 and 2 are sourced from natural or commercial sources and the desired formulation is formed as per manufacturing requirements (Step 402). Then, the aloe vera is blended and stained at a normal room temperature and pressure to make a uniform aloe vera liquid (Step 404). In the next step 406, all the organic oils are mixed to form the therapeutic composition. Finally, different testing techniques such as microbiological testing, sterilization, and filtering to remove any remains of the ingredients are performed. The testing is performed to maintain the purity of the composition (Step 408).

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “therapeutic scalp and skin care composition”, “scalp irritation prevention lotion”, “scalp treatment composition”, “hair and scalp treatment”, and “scalp and skin moisturizer composition” are interchangeable and refer to the therapeutic scalp and skin care composition 100 of the present invention.

Notwithstanding the forgoing, the therapeutic scalp and skin care composition 100 of the present invention can contain any additional ingredient known in the art without affecting the overall concept of the invention, provided that it accomplishes the above-stated objectives. One of ordinary skill in the art will appreciate that the ingredients and composition of the therapeutic scalp and skin care composition 100 as shown in the FIGS. are for illustrative purposes only, and that many other compositions are well within the scope of the present disclosure.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A scalp irritation prevention composition comprising:
a base of aloe vera, wherein the aloe vera is blended and filtered to create a smooth liquid;
a plurality of essential oils, wherein the essential oils is comprised of a peppermint oil, a lavender oil, a rosemary oil, a Virginia olive oil, an avocado oil, and a grapeseed oil; and
wherein the composition is formulated for topical application to the scalp and skin, and is configured to hydrate, soothe, and protect the scalp and skin from irritation.

2. The scalp irritation prevention composition of claim 1, wherein the aloe vera comprises 25% to 30% by weight of the scalp irritation prevention composition.

3. The scalp irritation prevention composition of claim 1, wherein the peppermint oil is present in an amount of 1% to 2% by weight.

4. The scalp irritation prevention composition of claim 1, wherein the lavender oil is present in an amount of 5% to 7% by weight.

5. The scalp irritation prevention composition of claim 1, wherein the rosemary oil is present in an amount of 1% to 3% by weight.

6. The scalp irritation prevention composition of claim 1, wherein the Virginia olive oil is present in an amount of 12% to 18% by weight.

7. The scalp irritation prevention composition of claim 1, wherein the avocado oil is present in an amount of 15% to 20% by weight.

8. The scalp irritation prevention composition of claim 1, wherein the grapeseed oil is present in an amount of 1% to 3% by weight.

9. The scalp irritation prevention composition of claim 1 further comprising sunflower oil in an amount of 10% to 15% by weight.

10. The scalp irritation prevention composition of claim 1 further comprising safflower oil in an amount of 5% to 8% by weight.

11. The scalp irritation prevention composition of claim 1 further comprising geranium oil in an amount of 2% to 3% by weight.

12. The scalp irritation prevention composition of claim 1 further comprising marjoram oil in an amount of 1% to 2% by weight.

13. The scalp irritation prevention composition of claim 1 further comprising lemongrass oil in an amount of 2% to 3% by weight.

14. The scalp irritation prevention composition of claim 1 further comprising tea tree oil.

15. The scalp irritation prevention composition of claim 14, wherein the tea tree oil is present in an amount of 1% to 2% by weight.

16. The scalp irritation prevention composition of claim 1 further comprising cedarwood oil.

17. The scalp irritation prevention composition of claim 16, wherein the cedarwood oil is present in an amount of 1% to 2% by weight.

18. The scalp irritation prevention composition of claim 7, wherein the avocado oil is comprised of an organic avocado oil.

19. A system for manufacturing a scalp irritation prevention composition comprising:
a blender configured to blend and filter a fresh aloe vera to form a smooth liquid;
a plurality of organic essential oils, wherein the organic essential oils are comprised of a peppermint oil, a lavender oil, a rosemary oil, a Virginia olive oil, an organic avocado oil, and a grapeseed oil; and
a mixer configured to mix the aloe vera liquid with the plurality of organic essential oils, wherein the system is configured to produce the composition for topical application to the scalp and skin.

20. A method of producing a scalp irritation prevention composition, the method comprising the following steps:
blending and filtering fresh aloe vera to form a smooth liquid;
mixing the aloe vera liquid with a plurality of organic essential oils, the organic essential oils comprised of a peppermint oil, a lavender oil, a rosemary oil, a Virginia olive oil, an organic avocado oil, and a grapeseed oil; and
forming the composition for topical application to the scalp and skin.
